(19) United States
(12) Patent Application Publication
(10) Pub. No.: US 2014/0194787 A1
(43) Pub. Date: Jul. 10, 2014

(54) INFRARED HEATING APPARATUS WITH FOOT MASSAGE FUNCTIONS

(71) Applicant: Globe Healthcare Corporation, Richmond (CA)

(72) Inventor: Mingyu XU, Tianjin (CN)

(73) Assignee: Globe Healthcare Corporation, Richmond (CA)

(21) Appl. No.: 14/070,197

(22) Filed: Nov. 1, 2013

(30) Foreign Application Priority Data
Jan. 8, 2013 (CA) 2801660

(51) Int. Cl. A61N 5/06

(52) U.S. Cl. CPC A61N 5/0625
USPC 601/15

(57) ABSTRACT

The present disclosure relates to an infrared heating apparatus. The apparatus comprises a housing having a hollow internal space, the housing comprising an opening through which a user's legs and feet may be inserted into the internal space of the housing. The apparatus comprises one or more heating panels located on inner sides of the housing for irradiating the user's legs. The apparatus comprises a massager located at a lower portion of the apparatus inside the internal space for massaging the user's feet.

### TECHNICAL FIELD

The present invention relates to infrared heating apparatus, and in particular infrared heating apparatus with foot massage functions.

### BACKGROUND

Sauna promotes the formation of perspiration (sweating) of the human body. Sauna use provides relaxation and certain health benefits. Conventionally, a sauna requires a room or a building especially built for that purpose. Building a sauna room or a sauna house is typically expensive.

Traditional massage has a history of more than a thousand years. Massage provides both relaxation and health benefits. Conventionally, massage is done by human hands. This can be expensive, as the hourly rate of a professional masseuse can be high. Also, manual massage is not always very effective, as the hands and arms of a masseuse can get tired easily.

### BRIEF DESCRIPTION OF DRAWINGS

In drawings which show non-limiting embodiments of the invention:

FIG. 1 is a perspective view of an infrared heating apparatus according to an example embodiment of the present invention.

FIG. 2 is a schematic perspective view of an infrared heating apparatus according to another example embodiment of the present invention.

FIG. 3 is an exploded view of the apparatus of FIG. 2.

FIG. 4 is a top perspective view of a massager which is installed in the apparatus of FIG. 2.

FIG. 5 is an exploded schematic view of the massager of FIG. 4.

FIG. 6 shows the massager of FIG. 4 being installed in the infrared heating apparatus.

FIG. 7 shows a worker installing a cover to cover the massager in the infrared heating apparatus.

FIG. 8 is a schematic diagram showing a person's legs and feet in a space in the infrared heating apparatus.

FIG. 8A is another schematic diagram showing a person's legs and feet in a space in the infrared heating apparatus.

FIG. 9 is a schematic diagram of example electrical circuits employed in the infrared heating apparatus according to an example embodiment.

FIG. 10 is a schematic sectional view of an example heating panel.

FIG. 11 is a schematic sectional view of an infrared light emitter located under a protrusion of a massager.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

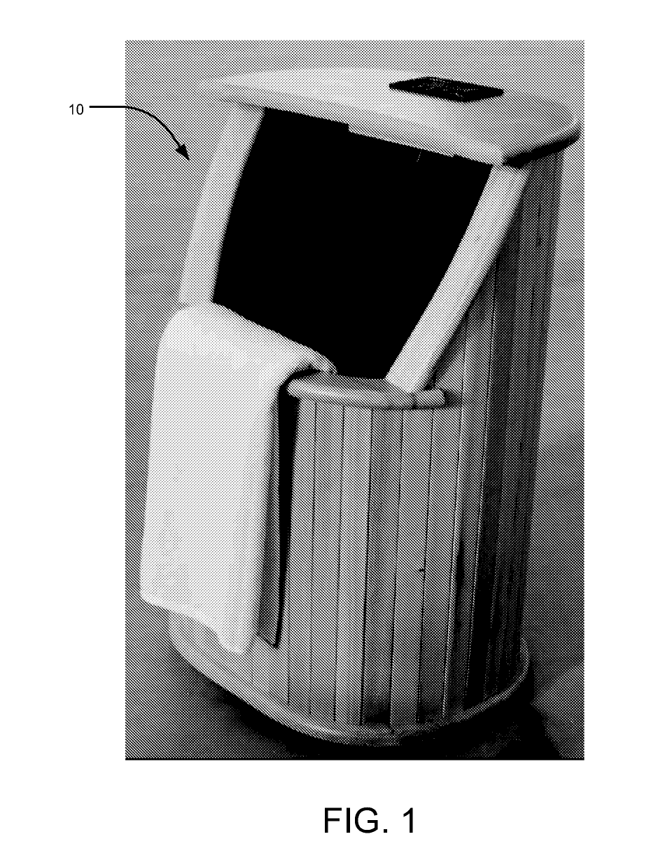

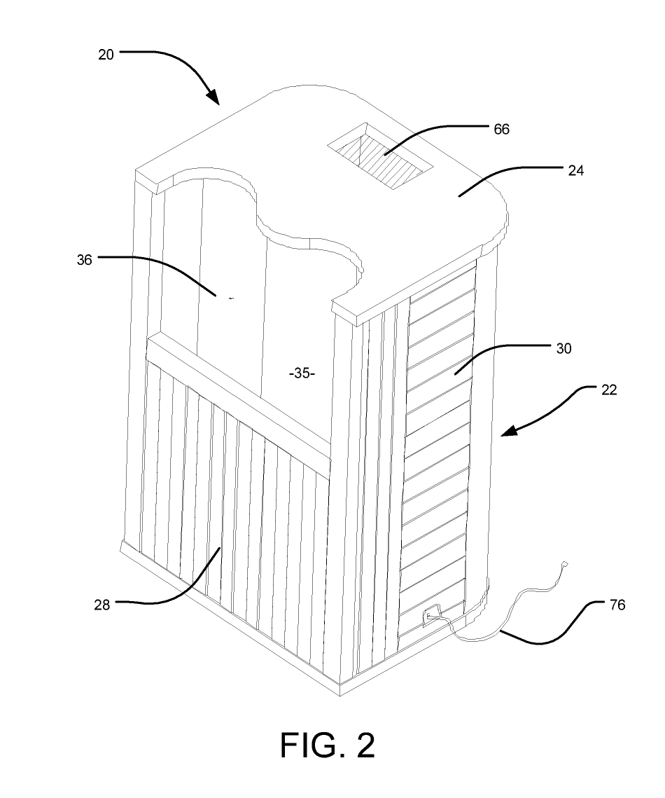

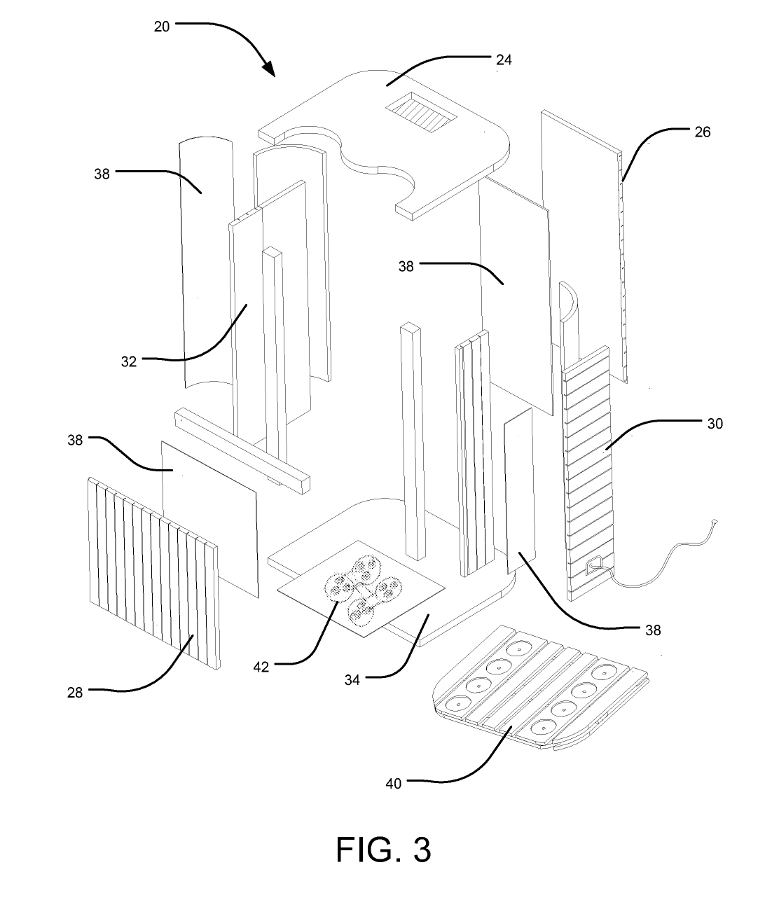

FIG. 1 is a perspective view of an infrared heating apparatus 10 according to an example embodiment of the present invention. FIG. 2 is a schematic perspective view of an infrared heating apparatus 20 according to another example embodiment of the present invention. FIG. 3 is an exploded view of apparatus 20.

Apparatus 20 comprises a housing 22. Housing 22 may have a cross-section that is round-shaped, or oval-shaped, or rectangular-shaped, or of some other suitable shape. In the embodiment of FIG. 2 (and FIG. 3), housing 22 comprises an upper cover board 24, a front cover board 26, a back cover board 28, a right cover board 30, a left cover board 32, and a bottom board 34. These boards together define housing 22 which has an internal hollow space 35. Housing 22 comprises an opening 36 through which a person's legs and feet can be inserted into space 35 of apparatus 20. The internal space 35 should be big enough to accommodate the person's both legs and both feet. In the embodiment in FIG. 2, opening 36 is bordered by upper cover board 24, back cover board 28, right cover board 30 and left cover board 32. Boards 24, 26, 28, 30 and 32 are made of an electrically non-conductive material (e.g., dry wood). This is a safety feature of apparatus 20.

The height and dimension of opening 36 should allow a user to easily insert his legs and feet (i.e., both left and right legs and both left and right feet) into space 35 of apparatus 20. For example, the height of back cover board 28 should be less than the knee height of a user. For example, the height of back cover board 28 may be in the range of 65% to 95%, or 70% to 90%, or 80% to 90% of the knee height of a user. The height of front cover board 26, right cover board 30, and left cover board 32 should be higher than the knee height of a user so that the user's knees do not contact the lower surface of upper cover board 24. For example, the height of front cover board 28 may be in the range of 105% to 130%, or 110% to 120% of the knee height of a user. In one particular embodiment, the dimensions of the infrared heating apparatus are 50 cm×40 cm×70 cm (wherein the height is 70 cm).

Apparatus 20 may comprise one or more heating panels 38. In some embodiment, apparatus 20 comprises a plurality of heating panels 38. In the illustrated embodiment of FIG. 3, heating panels 38 are located on the inner side of front cover board 26, back cover board 28, right cover board 30, and left cover board 32. This way, heating panels 38 would surround the user's legs. When turned on, heating panels 38 are capable of emitting infrared light to warm up a person's legs placed inside apparatus 20. In some embodiments, the heating panel may be a single continuous heating panel that extends around the inner side walls of the housing. In some embodiments, light in the far infrared range (e.g., 4 to 1000 microns) constitutes greater than 80% of the total light energy emitted by heating panels 38. In some embodiments, light in the range of 4 to 25 microns constitutes greater than 80% of the total light energy emitted by heating panels 38. In some embodiments, light in the range of 5.6 to 15 microns constitutes greater than 80% of the total light energy emitted by heating panels 38. When apparatus 20 is in use, the irradiation of a person's legs with infrared light in the far infrared range warms up the person's legs and provides relaxation. Unlike a conventional water bath which generally warms up the skin, the irradiation with infrared light in the far infrared range may penetrate the user's skin and warm up the tissue beneath the skin. Additionally, the irradiation with infrared light in the far infrared range may provide certain health benefits to the user, such as improving the body's microcirculation of blood, promoting metabolism, and improving human immunity.

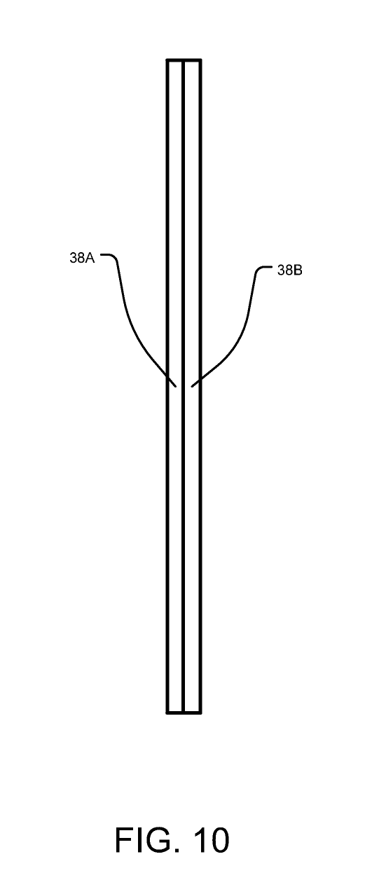

An example heating panel 38 is schematically shown in sectional view in FIG. 10. In the embodiment of FIG. 10, heating panel 38 comprises a first layer 38A and a second layer 38B. First layer 38A faces toward internal space 35 of apparatus 20, whereas second layer 38B is proximal to housing 22 of apparatus 20. First layer 38A comprises a substrate (e.g., cloth, non-woven fabric, or a polymer material) infused with an infrared heating composition. In one particular embodiment, first layer 38 is made of non-woven fabric soaked with the infrared heating composition. The infrared heating composition may comprise an infrared carbon paste composition. The infrared carbon paste composition comprises carbon paste mixed with an auxiliary substance, such as a metal or non-metal oxide (e.g., zinc oxide, aluminum oxide, germanium oxide, manganese oxide, copper oxide, zirconium dioxide, or some other suitable material). In some embodiments, the auxiliary substance constitutes 10% to 20% by weight of the infrared carbon paste composition, whereas carbon paste constitutes 80% to 90% by weight of the infrared carbon paste composition. Second layer 38B comprises a polymer layer (e.g., a resin layer) which is resistant to heat and high temperature. Second layer 38B also comprises electric wires, a fuse, and a connection to a control switch. Second layer 38B may also comprise copper and/or metal foil.

Apparatus 20 may optionally comprise a base board 40. Base board 40 may be placed inside apparatus 20 on top of bottom board 34. Base board 40 may function to facilitate the installation of a massager in apparatus 20.

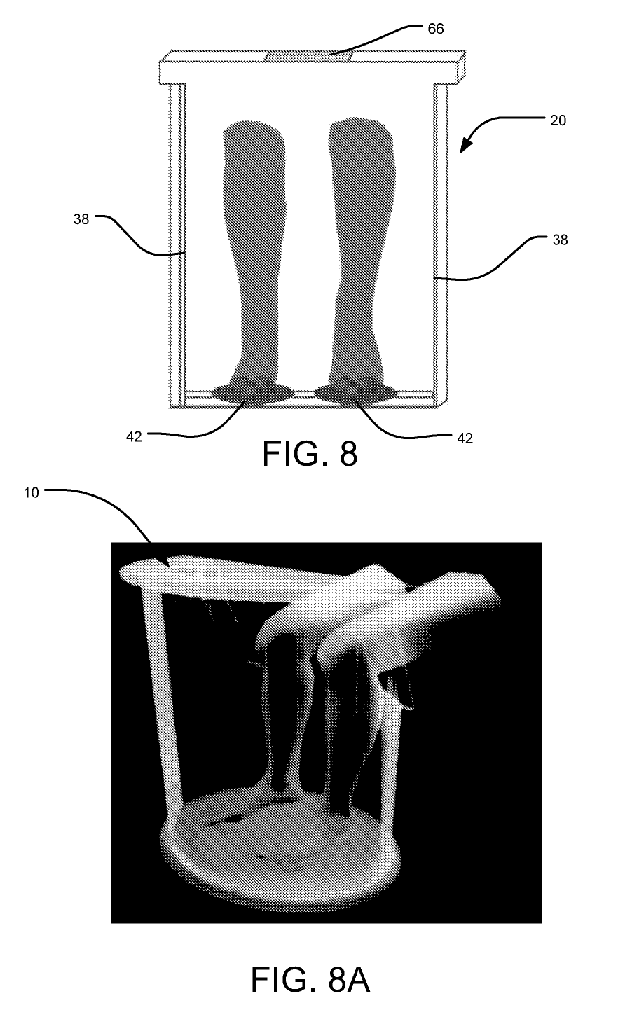

Apparatus 20 comprises a massager 42. Massager 42 is located inside apparatus 20 in a lower portion thereof and sits on top of bottom board 34 or base board 40. In use, a person inserts his or her legs and feet into space 35 of apparatus 20 and rests his or her feet on massager 42. When the massager 42 is turned on, massager 42 massages the lower surfaces of the person's feet. At the same time, the person's entire legs are warmed up by heating panels 38 which circumferentially surround the person's legs. This is schematically illustrated in FIGS. 8 and 8A.

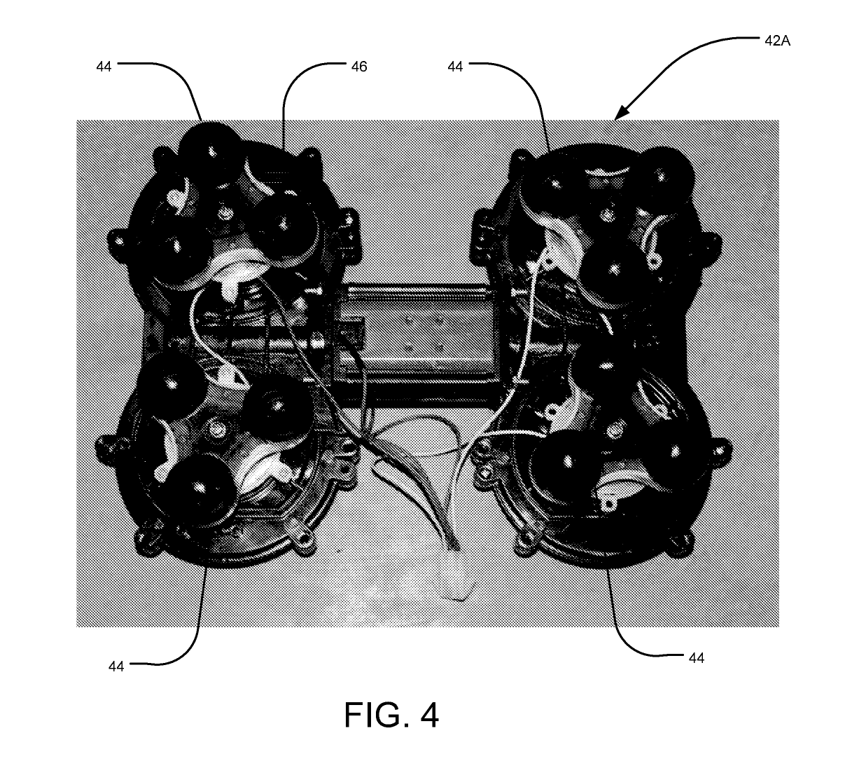

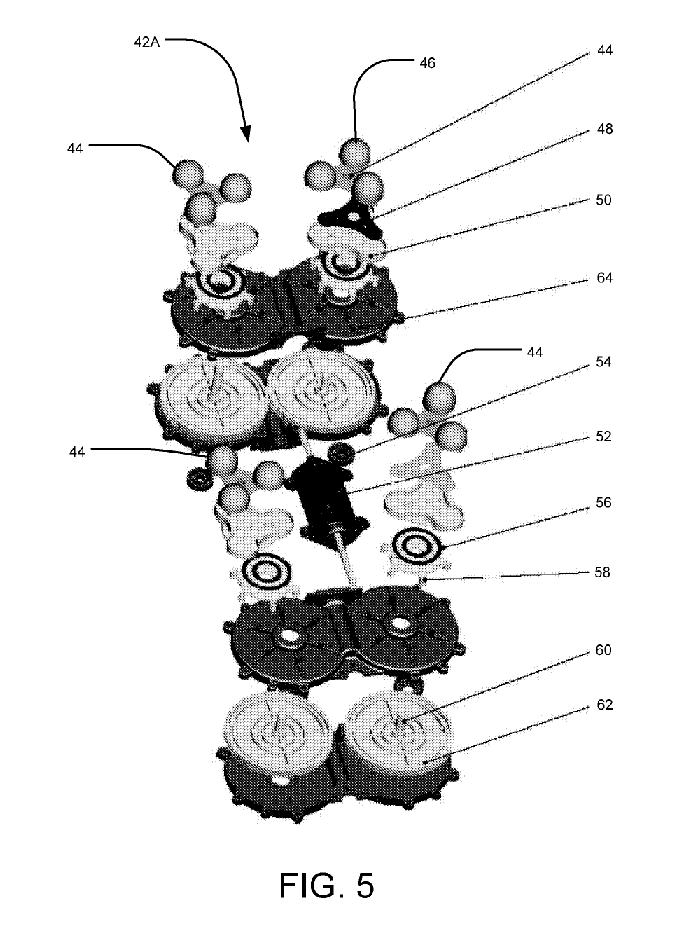

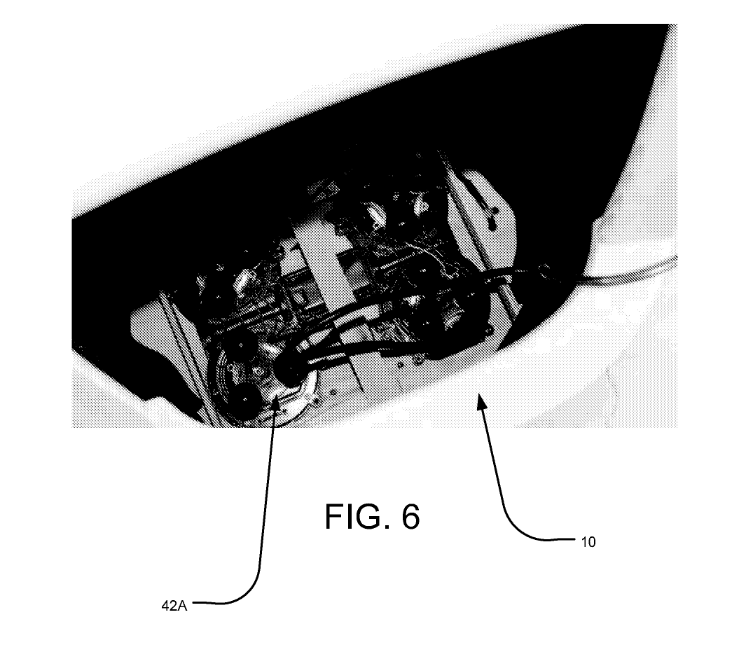

FIG. 4 is a top perspective view of an example massager 42A shown in isolation. FIG. 5 is an exploded schematic view of massager 42A. FIG. 6 shows massager 42A being installed in apparatus 10. Massager 42A is a mechanical massager that uses mechanical force to massage a user's feet. No fluid or liquid is used with massager 42A. Unlike a foot bath/spa, the user's feet are not submerged in water. Massager 42A comprises four massage heads 44. Two of massage heads 44 are disposed on a left portion of massager 42A and the other two of massage heads 44 are disposed on a right portion of massager 42A so that two massage heads 44 can massage a person's left foot and the other two massage heads 44 can massage a person's right foot. In the illustrated embodiment, each massage head 44 comprises three semi-spherical massage protrusions 46, although this is not mandatory. It is possible to have one, two, or other suitable number of massage protrusions 46 on a massage head 44. Massage protrusions 46 are made of an electrically non-conductive material. When massager 42A is turned on, massage heads 44 are able to move in a translational or rotational or translational/rotational fashion to massage a person's feet. For example, the translational movement of massage heads 44 may be on a plane that is generally parallel to the lower surfaces of the user's feet.

As illustrated in FIG. 5, each massage head 44 is coupled to a massage head circuit board 48 located on a base 50. Massager 42A comprises a motor 52 which drives the movement of massage head 44. Motor 52 is operatively coupled to massage head 42 via components such as bearing 54, conducting sheet 56, electric brush plate 58, gear shaft 60, and gear 62. Each gear 62 is covered by a gear cover case 64.

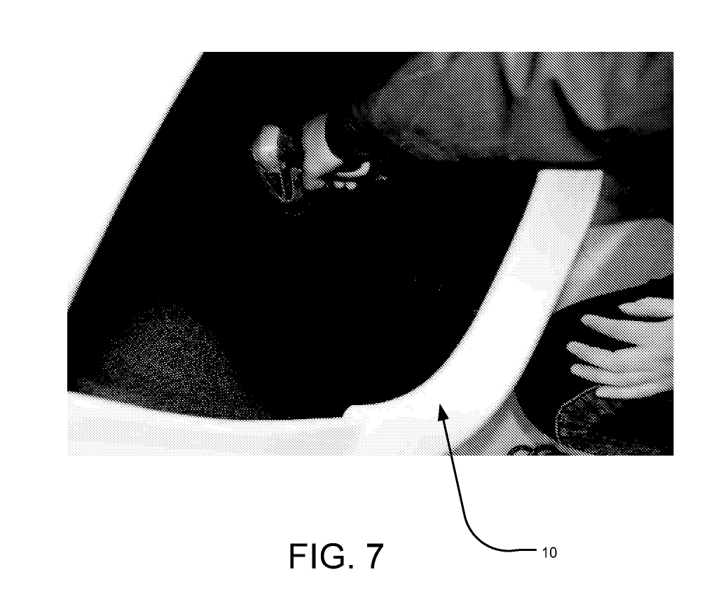

To provide more comfort to the user, massager 42A may comprise a cover or cushion. FIG. 7 shows a worker installing a cover or cushion on massager 42A.

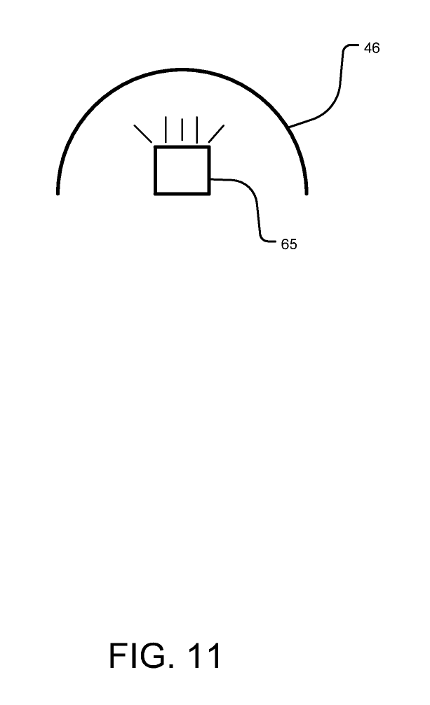

In some embodiments, infrared light emitters 65 are provided in one or more massage protrusions 46. This is schematically illustrated in FIG. 11. Infrared light emitters 65 are capable of emitting infrared light to warm up a person's feet placed on massager 42. In some embodiments, light in the far infrared range (e.g., 4 to 1000 microns) constitutes greater than 80% of the total light energy emitted by infrared light emitters 65. In some embodiments, light in the range of 5.6 to 15 microns constitutes greater than 80% of the total light energy emitted by infrared light emitters 65. In embodiments with infrared light emitters 65, protrusions 46 may be made of a material that is permeable to infrared light. In such embodiments, it is either not necessary to cover massager 42 with a cover or cushion, or such cover or cushion is made of a material that is permeable to infrared light.

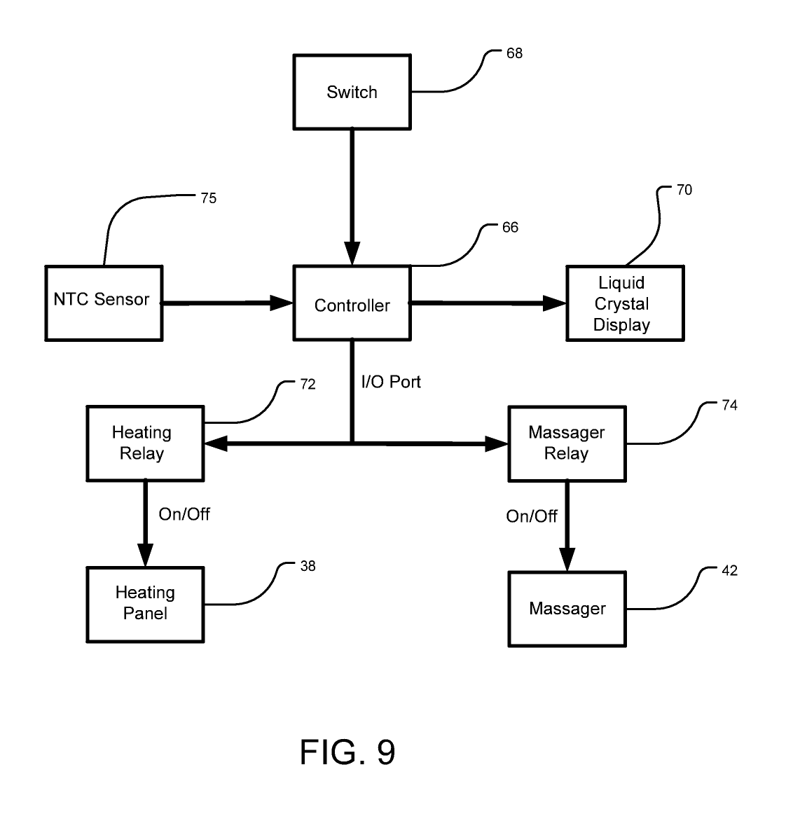

FIG. 9 is a schematic diagram of example electrical circuits employed in apparatus 20. Apparatus 20 may comprise a controller 66. Controller 66 may be controlled or adjusted by a user by using one or more switches 68. Switches 68 may enable the user to turn on or off heating panels 38 and/or massager 42, or to set or adjust a desired temperature or temperature range for heating panels 38, or to set or adjust the speed of massage heads 44 of massager 42. Controller 66 may be coupled to a liquid crystal display 70. Display 70 may display information such as the on/off status of heating panels 38 and/or massager 42, the set temperature or temperature range for heating panels 38, the current temperature inside apparatus 20, the speed or setting of massage heads 44, etc. Controller 66 is electrically coupled to heating panels 38 via a heating relay 72, and to massager 42 via a massager relay 74. In some embodiments, controller 66, switches 68, and/or display 70 may be disposed on upper cover board 24.

In some embodiments, apparatus 20 comprises an NTC (negative temperature coefficient) sensor 75. NTC sensor 75 may be located at an upper portion of space 35 of apparatus 20, near upper cover board 24. NTC sensor is used to measure temperature in space 35 of apparatus 20 and provides the temperature information to controller 66 to automatically control (e.g., turn on or turn off) heating panels 38 to achieve a desired temperature or temperature range.

Controller 66 of apparatus 20 allows a user to set a maximum heating temperature below a certain point so that when the measured temperature exceeds the set point heat panels 38 can be automatically turned off. For example, a user may set the maximum heating temperature of apparatus 20 to be below 60° C. The temperature may be monitored by a temperature sensor (e.g., NTC sensor 75). A user may also set the heating temperature of apparatus 20 within a range (e.g., between 40° C. and 60° C.). The inventor has empirically determined that a heating temperature range between 40° C. and 60° C. (e.g., between 40° C. and 50° C.) provides a high level of comfort and is also generally safe for the user.

Apparatus 20 may comprise a power input line 76 (see FIG. 2). Power input line 76 connects apparatus 20 to a power source. Alternatively, apparatus 20 may be operated by batteries.

The apparatus according to the present disclosure has a number of advantages. It is portable, and can be moved from room to room. The apparatus is a water-free and liquid-free apparatus. It does not use water (or other liquid), which is safer than a conventional water bath/spa as water is a conductor of electricity. Also, no water will be spilled on the floor if it is used in a living room or an office. It is not very expensive to construct. It warms up a person's legs and at the same time massages the person's feet. Because the heating panel(s) surround the person's legs, it warms up the person's entire legs, not just a small localized area of one of the legs. It improves microcirculation in the user's legs and feet, eases pain in the lower limbs and removes fatigue. Additionally, a user may sit comfortably and do other useful things (e.g., reading books or newspapers or working on a computer) while his or her legs and feet are being treated by the infrared heating apparatus according to the present disclosure. The simultaneous heating and massaging of the user's feet is especially advantageous. According to traditional Chinese medicine, many important pressure and acupuncture points are located at the bottom of a person's feet. There pressure points are thought to be connected to important internal organs of the human body via a network of channels in which a life-energy known as “qi” flows. Stimulating these pressure points on the feet and warming up the legs and feet at the same time to increase blood supply and “qi” supply to these regions of pressure points have synergistic effects and may provide enhanced health benefits to the user than foot massaging alone or heating alone.

The scope of the claims should not be limited by the preferred embodiments set forth in the examples, but should be given the broadest interpretation consistent with the description as a whole.

What is claimed is:

1. An apparatus comprising:
a housing having a hollow internal space for accommodating a user's legs and feet, the housing comprising an opening through which the user's legs and feet may be inserted into the internal space of the housing;
one or more heating panels located circumferentially on inner sides of the housing to surround the user's legs for irradiating the user's legs;
a massager located at a lower portion of the apparatus inside the internal space for mechanically massaging the user's feet.

2. An apparatus according to claim 1, wherein the housing comprises an upper cover board, a front cover board, a back cover board, a right cover board, a left cover board, and a bottom board.

3. An apparatus according to claim 2, wherein the one or more heating panels are located on the inner sides of the front cover board, the back cover board, the right cover board, and the left cover board.

4. An apparatus according to claim 1, wherein the heating panels are capable of emitting infrared light.

5. An apparatus according to claim 4, wherein the heating panels are capable of emitting infrared light in a far infrared range.

6. An apparatus according to claim 5, wherein the heating panels are capable of emitting infrared light in a range of 5.6 to 15 microns and the infrared light in the range of 5.6 to 15 microns constitutes greater than 80% of the total light energy emitted by the heating panels.

7. An apparatus according to claim 6, wherein the infrared light in the range of 5.6 to 15 microns constitutes greater than 90% of the total light energy emitted by the heating panels.

8. An apparatus according to claim 1, wherein the one or more heating panels generate a heating temperature in a range between 40° C. and 60° C.

9. An apparatus according to claim 1, wherein the one or more heating panels generate a heating temperature in a range between 40° C. and 50° C.

10. An apparatus according to claim 1, wherein at least one of the one or more heating panels comprises a first layer and a second layer.

11. An apparatus according to claim 10, wherein the first layer comprises non-woven fabric infused with an infrared heating composition.

12. An apparatus according to claim 11, wherein the infrared heating composition comprises carbon paste mixed with an auxiliary substance, wherein the auxiliary substance comprises zinc oxide, aluminum oxide, germanium oxide, manganese oxide, copper oxide, or zirconium dioxide.

13. An apparatus according to claim 12, wherein the auxiliary substance constitutes 10% to 20% by weight of the infrared heating composition and the carbon paste constitutes 80% to 90% by weight of the infrared heating composition.

14. An apparatus according to claim 11, wherein the second layer comprises a resin layer.

15. An apparatus according to claim 1, wherein the massager comprises four massage heads, with two massage heads for massaging the left foot and two massage heads for massaging the right foot.

16. An apparatus according to claim 15, wherein each one of the massage heads comprise two, three, or four massage protrusions.

17. An apparatus according to claim 16, wherein an infrared light emitter is provided under each one of the massage protrusions.

18. An apparatus according to claim 17, wherein the massage protrusions are made of a material permeable to infrared light.

19. An apparatus according to claim 1, comprising a controller which controls the massager and the heating panels.

20. An apparatus according to claim 1, wherein the apparatus comprises a temperature sensor for sensing the temperature in the internal space of the apparatus.
